(19) United States
(12) Plant Patent Application Publication
(10) Pub. No.: US 2002/0152529 P1
(43) Pub. Date: Oct. 17, 2002

(54) Lily plant named 'Yelloween'

(75) Inventor: Peter Jan Kos, Anna Paulowna (NL)

Correspondence Address: KLARQUIST SPARKMAN, LLP, 121 SW SALMON STREET, SUITE 1600, PORTLAND, OR, 97204, US

(73) Assignee: World Breeding, B.V.

(21) Appl. No.: 09/834,758

(22) Filed: Apr. 13, 2001

(51) Int. Cl. A01H 5/00

(52) U.S. Cl. PLT/313

(57) ABSTRACT

A new and distinct variety of upright Oriental×Aurelia hybrid lily plant, named ‘Yelloween’, characterized by large, upright, yellow flowers.

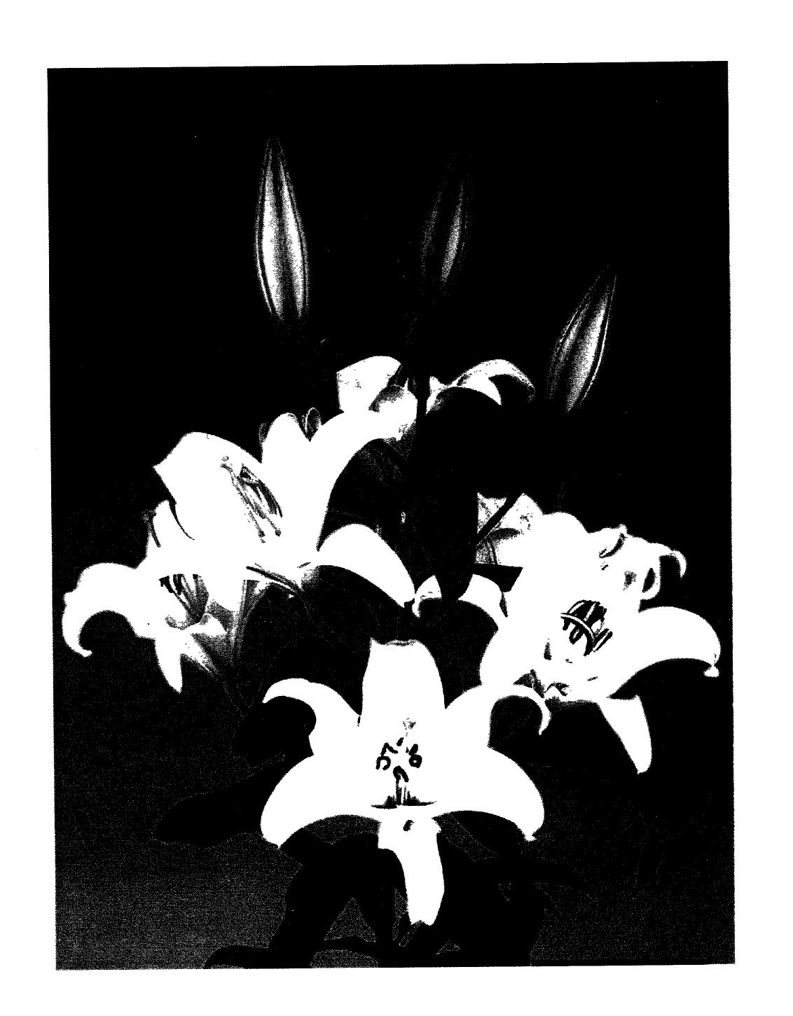

The present invention relates to a new and distinct variety of Lilium hybrid referred to commercially as an upright Oriental x Aurelia hybrid lily and given the cultivar name ‘Yelloween’.

This new variety is the product of a controlled breeding program using embryo culture and was carried out in the municipality of Breezand, The Netherlands, using the proprietary Oriental type hybrid lily 91.30.00-2 as the seed parent and the proprietary Aurelia type hybrid lily 90.058-1 as the pollen parent. The resulting plants were evaluated for cut flower production. Compared to the typical characteristics of common Oriental and Aurelia lily hybrids, ‘Yelloween’ has larger flowers, taller plant height and taller stems, and blooms earlier.

This new variety has not been observed under all possible environmental conditions. The phenotype may vary significantly with variations in environment such as temperature, light intensity, day length, and nutrient availability, without, however, any variations in genotype.

The following traits have been repeatedly observed and are determined to be the basic characteristics of this invention that, in combination, distinguish this lily as a new and distinct variety:

1. floriferous habit;

2. large upright yellow flowers;

3. vigorous growth habit;

4. tall plant;

5. good virus tolerance; and

6. excellent vase-life.

Bulbs of this plant may be pre-cooled and forced out of season for very uniform cut flower production.

The primary distinguishing feature of ‘Yelloween’ is its numerous, large, upright, yellow flowers.

Asexual reproduction of this new variety has been accomplished at a nursery in Breezand, The Netherlands, by scaling and tissue culture. The resulting plants have shown that the above-mentioned unique features are stable and reproduce true to type in each successive propagation.

### BRIEF DESCRIPTION OF THE DRAWING

This new variety of lily plant is illustrated in the accompanying photographic drawing which shows the upper half portion of the flowering stem of a potted plant in the full face view of the flowers together with some foliage. The accompanying photograph, showing ‘Yelloween’, was taken in a studio and illustrates the typical flower and foliage form, with color as true as reasonably possible in this type of color illustration.

### DETAILED DESCRIPTION

This detailed description of the ‘Yelloween’ variety is based on plants produced in greenhouses in Breezand, The Netherlands, with color references according to the RHS Colour Chart published by The Royal Horticultural Society of London, England, except where the context indicates a term having its ordinary dictionary meaning.

Origin: Resulting from a hybridization.

Parentage:

Seed Parent.—Oriental type hybrid lily 91.30.00-2 (proprietary owned).

Pollen Parent.—Aurelia type hybrid lily 90.058-1 (proprietary owned).

Commercial classification: Upright Oriental x Aurelium hybrid lily, cut flower type.

Classification: Division VIII, all hybrids not provided for in any previous Division according to the Horticultural Classification of Lilies by The Royal Horticultural Society.

Form: Single erect stem from each bulb bearing terminal cluster of flowers.

Height.—To about 160 cm. from the top of the soil to the top of the inflorescence from bulbs of about 32 cm in circumference.

Stem: Color is Green Group (RHS 138B) ascending to Green Group (RHS 137D).

Growth: Vigorous, upright, and strong.

Foliage:

Arrangement.—Gradual.

Quantity.—Many fine leaves.

Leaf size.—Variable, lower leaves on the stem about 10.5 cm. long and about 4 cm. wide; upper leaves on the stem about 19.5 cm long and about 3 cm wide.

Leaf shape.—Lower leaves: Lanceolate. Upper leaves: Round top and wide-shaped.

Leaf margin.—Entire.

Aspect.—Satiny.

Texture.—Leathery.

Color.—Upperside of leaves: Green (RHS 139A). Underside of leaves: Green (RHS 138B).

Bud:

Size.—To about 12 cm. long and to about 3 cm. wide, just prior to opening.

Shape.—Lanceolate, with the apex commonly having three obtuse tips.

Color.—At first opening, the color is Yellow (RHS 5C), turning Yellow (RHS 6A) when unfurling.

Opening Rate.—The mature bud opens slowly in response to light.

Flower:

Blooming habit.—Annually, once and profusely, early when planted outdoors and year round when planted in a greenhouse.

Days to bloom.—Flowers approximately 80 days after greenhouse planting under typical flower forcing conditions at minimum temperature of 15° C.

Flower size.—Large.

Diameter.—To about 23 cm.

Depth.—To about 7 cm.

Borne.—Raceme of about 3 flowers from bulbs of about 12 to 14 cm. in circumference.

Shape.—The flower is funnel shaped, later the funnel flattens out.

Tepalage.—Number: 6.

Arrangement.—Imbricated.

Shape.—Oval.

Texture.—Leathery.

Aspect.—Glossy.

Size.—Length: Inner tepals to about 14 cm. Outer tepals to about 14.3 cm. Width: Inner tepals to about 6 cm. Outer tepals to about 4 cm.

Nectary.—Light Green (RHS 144A) from base. Oozing to about 2 cm.

Spots.—None.

Blush.—None.

Color.—Outer tepals: Bottomside: Yellow (RHS 6A). Upperside: Yellow (RHS 5C). Inner tepals: Bottomside: Yellow (RHS 6A). Upperside: Yellow (RHS 5C).

Fragrance.—Weak.

Lasting Quality.—Excellent. Each flower lasts about 14 days, depending upon environmental variables.

Orientation.—Ascending upwardly off the stem, the first flower at about a 30° angle to about a 35° angle.

Pedicel.—Length: About 14 cm. Color: Green (RHS 137C).

Reproductive Organs:

Stamens.—Number: Six, typical of the genus Lilium.

Anthers before dehiscense.—Size: To about 4.3 cm. from mature bud. Color: Dark Purple (RHS 187A).

Filaments.—Length: To about 8.4 cm. Color: Yellow-Green (RHS 145C).

Pollen.—Color: Brown (RHS 183A).

Pistil.—Number: One.

Style.—Length: About 8 cm. Color: Light Green (RHS 145C).

Stigma.—Color: Light Green (RHS 149C).

Fruit:

Fertility.—The fruit is fertile.

Shape.—Ovoid.

Color at maturity.—Brown Green (RHS 152B).

I claim:

1. A new and distinctive variety of Oriental×Aurelia hybrid lily plant, substantially as herein illustrated and described:
